(19) United States
(12) Patent Application Publication
(10) Pub. No.: US 2005/0220740 A1
(43) Pub. Date: Oct. 6, 2005

(54) Cosmetic composition comprising concave particles

(76) Inventor: Christophe Dumousseaux, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/085,519

(22) Filed: Mar. 22, 2005

Related U.S. Application Data
(60) Provisional application No. 60/554,931, filed on Mar. 22, 2004.

(30) Foreign Application Priority Data
Mar. 22, 2004 (FR) 04 50560

(51) Int. Cl. A61K 7/025

(52) U.S. Cl. 424/64

(57) ABSTRACT

A cosmetic composition comprising a pulverulent phase and a liquid fatty phase is disclosed, wherein the pulverulent phase comprises pigments and concave particles. The pigments are present in an amount of less than or equal to 15% by weight relative to the total weight of the composition, and the liquid fatty phase is present in an amount of less than or equal to 13% by weight relative to the total weight of the composition. The present disclosure also relates to a process for making up and to caring for keratinous substances, such as the skin, comprising the application of the composition to the keratinous substances.

This application claims benefit of U.S. Provisional Application No. 60/554,931, filed Mar. 22, 2004, and French Patent Application No. 04/50560, filed Mar. 22, 2004, both of which are hereby incorporated by reference.

The present disclosure relates to a cosmetic composition, such as a composition in the form of a compact powder, comprising pigments and concave particles. The present disclosure also relates to a cosmetic process for making up or caring for human keratinous substances, such as the skin, hair or nails.

The composition disclosed herein can be a composition for making up or caring for the skin and can be provided in the form of a blusher, an eyeshadow, a face powder, a foundation, a concealer, a product for making up the body, a product for caring for the face, a product for caring for the body or an antisun product. In one embodiment, the present disclosure relates to a foundation composition.

Make-up powders generally comprise, on the one hand, a pulverulent phase comprising, for example, pigments and fillers and, on the other hand, a fatty phase as binder comprising fatty substances. The fatty phase is intended to confer a degree of density on the finished product, to bestow softness and an emollient property on the make-up product and to promote its adherence to the skin.

Some make-up compositions, such as foundations, eyeshadows and blushers, are provided in the form of a compact powder generally comprising a fatty phase, referred to as binder, and a pulverulent phase comprising in particular pigments and/or fillers.

The preparation of compact powders raises numerous difficulties as the final composition has to be sufficiently homogeneous and compact to prevent fragmentation of the product brought about in particular by impacts. The compact powder must also be easy to remove in order to allow the user to remove the necessary amount of product in order to apply it subsequently to keratinous substances, such as the skin.

Concave particles formed of crosslinked organopolysiloxane material which have the shape of portions of hollow spheres, obtained by condensation of silanols resulting from the hydrolysis of organosilicone compounds, are known from Japanese Patent Application Nos. JP-A-2000-191789 and JP-A-2003-128788.

In these applications, a suggestion is made to use the concave particles in cosmetic products for the face or in make-up products, such as in compact foundation powders. These make-up products confer softness during their application to the skin and adhere well to the skin. These patent applications also teach the formulation of these particles in compact powders in the presence of 16% or 20% by weight of oils and 15% by weight of titanium dioxide or 25% by weight of pulverulent coloring materials. However, such compact powders have the disadvantage of exhibiting a poor ability to be removed, they disintegrate with difficulty using a finger or using a sponge, and the user does not succeed in taking a sufficient amount of product to be able to suitably apply the makeup. Furthermore, this difficulty in disintegrating the product often prompts the user to vigorously rub over the surface of the compact powder in order to succeed in taking a larger amount of product, but the more vigorous rubbing causes the surface of the product to harden, rendering the surface smooth. The product then becomes even more difficult to disintegrate. Furthermore, the properties of softness, and of spreading are difficult to perceive by the user during the application of the product to the skin.

It would thus be desirable to have available a cosmetic composition, such as make-up, comprising concave particles which can spread easily over keratinous substances, such as over the skin, and which, when it is provided in the form of a compact powder, can readily disintegrate with a finger or using a sponge.

The present inventors have discovered that such a composition is obtained by using a reduced content of liquid binder and of pigments with the concave particles. The composition spreads well over the skin and exhibits good slip, facilitating satisfactory distribution of the product over the surface of the skin and thus makes it possible to obtain a make-up homogeneously distributed over the skin. Furthermore, when the composition is in the form of a compact powder, the latter then exhibits good disintegrating properties, allowing the user to readily take up, with a finger or with a sponge, the necessary amount of product to apply the make-up.

More specifically, the present disclosure relates to a cosmetic composition, such as a compact powder, comprising a pulverulent phase and a liquid fatty phase, the pulverulent phase comprising pigments and concave particles, such as particles in the form of portions of hollow spheres composed of a material such as an organosilicone material, the pigments being present in a content of less than or equal to 15% by weight relative to the total weight of the composition, and the liquid fatty phase being present in a content of less than or equal to 13% by weight relative to the total weight of the composition.

Another aspect of the present disclosure is a cosmetic process for making up or for the non-therapeutic care of keratinous substances, such as the skin, comprising the application to the keratinous substances of a composition as disclosed herein.

The composition according to the present disclosure comprises concave particles. These particles thus have a surface exhibiting a rounded interior.

In one embodiment, the concave particles are particles in the form of portions of hollow spheres composed of a material such as an organosilicone material.

The particles beneficially have a mean diameter ranging from 0.05 μm to 10 μm.

The portions of hollow spheres used in the composition according to an embodiment of the present disclosure can have the shape of truncated hollow spheres exhibiting a single orifice communicating with their central cavity and having a transverse cross section with the shape of a horseshoe or arch.

The organosilicone material of the concave particles can be a crosslinked polysiloxane with a three-dimensional structure. In one embodiment, the crosslinked polysiloxane with a three-dimensional structure comprises units of formula (I): SiO₂, and of formula (II): R¹SiO_{1.5}, wherein R¹ comprises an organic group having a carbon atom directly connected to the silicon atom. The organic group can be chosen from a reactive organic group and an unreactive organic group. In one embodiment of the present disclosure, the organic group is an unreactive organic group.

The unreactive organic group can be a C₁-C₄ alkyl group, such as a methyl, ethyl, propyl or butyl group, or a phenyl group. In one embodiment of the present disclosure, the unreactive organic group is a methyl group.

The reactive organic group can be chosen from an epoxy group, a (meth)acryloyloxy group, an alkenyl group, a mercaptoalkyl group, an aminoalkyl group, a haloalkyl group, a glyceroxy group, an ureido group and a cyano group. In one embodiment of the present disclosure, the reactive organic group can be chosen from an epoxy group, a (meth)acryloyloxy group, an alkenyl group, a mercaptoalkyl group and an aminoalkyl group. The reactive organic group generally comprises from 2 to 6 carbon atoms, such as from 2 to 4 carbon atoms.

Among the epoxy groups that can be used, non-limiting mention may be made of a 2-glycidoxyethyl group, a 3-glycidoxypropyl group or a 2-(3,4-epoxycyclohexyl)propyl group.

Among the (meth)acryloyloxy groups that may be used, non-limiting mention may be made of a 3-methacryloyloxypropyl group or a 3-acryloyloxypropyl group.

Among the alkenyl groups that may be used, non-limiting mention may be made of a vinyl group, an allyl group or an isopropenyl group.

Among the mercaptoalkyl groups that may be used, non-limiting mention may be made of a mercaptopropyl group or a mercaptoethyl group.

Among the aminoalkyl groups that may be used, non-limiting mention may be made of a 3-[(2-aminoethyl)amino]propyl group, a 3-aminopropyl group or an N,N-dimethylaminopropyl group.

Among the haloalkyl groups that may be used, non-limiting mention may be made of a 3-chloropropyl group or a trifluoropropyl group.

Among the glyceroxy groups that may be used, non-limiting mention may be made of a 3-glyceroxypropyl group or a 2-glyceroxyethyl group.

Among the ureido groups that may be used, non-limiting mention may be made of a 2-ureidoethyl group.

Among the cyano groups that may be used, non-limiting mention may be made of a cyanopropyl group or a cyanoethyl group.

In one embodiment of the present disclosure, in the unit of formula (II), R¹ denotes a methyl group.

In one embodiment of the present disclosure, the organosilicone material comprises the units (I) and (II) according to a unit (I)/unit (II) molar ratio ranging from 30/70 to 50/50. In a further embodiment of the present disclosure, the unit (I)/unit (II) ratio may range from 35/65 to 45/55.

The organosilicone particles can be capable of being obtained according to a process comprising:

(a) introducing into an aqueous medium, in the presence of at least one hydrolysis catalyst and optionally of at least one surfactant, a compound (III) of formula SiX₄ and a compound (IV) of formula RSiY₃, wherein

X and Y are chosen from, independently of one another, a C₁-C₄ alkoxy group, an alkoxyethoxy group including a C₁-C₄ alkoxy group, a C₂-C₄ acyloxy group, an N,N-dialkylamino group including a C₁-C₄ alkyl group, a hydroxyl group, a halogen atom and a hydrogen atom, and

R is an organic group comprising a carbon atom connected directly to the silicon atom; and

(b) bringing the mixture resulting from stage (a) into contact with an aqueous solution including at least one polymerization catalyst and optionally at least one surfactant, at a temperature of between 30 and 85° C., for at least two hours.

Stage (a) corresponds to a hydrolysis reaction and stage (b) corresponds to a condensation reaction.

In stage (a), the molar ratio of the compound (III) to the compound (IV) generally ranges from 30/70 to 50/50. In one embodiment of the present disclosure, the molar ratio of compound (III) to compound (IV) ranges from 35/65 to 45/45. In a further embodiment of the present disclosure, the molar ratio of compound (III) to compound (IV) is 40/60. The ratio by weight of the water to the total weight of the compounds (III) and (IV) can range from 10/90 to 70/30. The order of introduction of the compounds (III) and (IV) generally depends on their rate of hydrolysis. The temperature of the hydrolysis reaction generally ranges from 0 to 40° C. and usually does not exceed 30° C. in order to prevent premature condensation of the compounds.

For the X and Y groups of the compounds (III) and (IV), non-limiting mention may be made of the following groups:

C₁-C₄ alkoxy groups such as the methoxy or ethoxy groups;

alkoxyethoxy groups including a C₁-C₄ alkoxy group, such as the methoxyethoxy or butoxyethoxy groups;

C₂-C₄ acyloxy groups such as the acetoxy or propionyloxy groups;

N,N-dialkylamino groups including a C₁-C₄ alkyl group, such as the dimethylamino or diethylamino groups; and

halogen atoms such as the chlorine or bromine atoms.

Among the compounds of formula (III) that may be used according to the present disclosure, non-limiting mention may be made of tetramethoxysilane, tetraethoxysilane, tetrabutoxysilane, trimethoxyethoxysilane, tributoxyethoxysilane, tetraacetoxysilane, tetrapropioxysilane, tetra(dimethylamino)silane, tetra(diethylamino)silane, silanetetraol, chlorosilanetriol, dichlorodisilanol, tetrachlorosilane or chlorotrihydrosilane. In one embodiment of the present disclosure, the compound of formula (III) is chosen from tetramethoxysilane, tetraethoxysilane, and tetrabutoxysilane, and mixtures thereof.

The compound of formula (III) results, after the polymerization reaction, in the formation of the units of formula (I).

The compound of formula (IV) results, after the polymerization reaction, in the formation of the units of formula (II).

The R group in the compound of formula (IV) has the meaning as described for the R¹ group for the compound of formula (II).

Among examples of compounds of formula (IV) comprising an unreactive organic group R, non-limiting mention may be made of methyltrimethoxysilane, ethyltriethoxysilane, propyltributoxysilane, butyltributoxysilane, phenyltri-methoxyethoxysilane, methyltributoxyethoxysilane, methyltriacetoxysilane, methyltripropioxysilane, methyltri(dimethylamino)silane, methyltri(diethylamino)silane, methylsilanetriol, methylchlorodisilanol, methyltrichlorosilane or methyltrihydrosilane.

As examples of compounds of formula (IV) comprising a reactive organic group R, non-limiting mention may be made of:

silanes having an epoxy group, such as (3-glycidoxypropyl)trimethoxysilane, (3-glycidoxypropyl)triethoxysilane, [2-(3,4-epoxycyclohexyl)ethyl]trimethoxysilane, (3-glycidoxypropyl)methyldimethoxysilane, (2-glycidoxyethyl)methyldimethoxysilane, (3-glycidoxypropyl)dimethylmethoxysilane or (2-glycidoxyethyl)dimethylmethoxysilane;

silanes having a (meth)acryloyloxy group, such as (3-methacryloyloxypropyl)trimethoxysilane or (3-acryloyloxypropyl)trimethoxysilane;

silanes having an alkenyl group, such as vinyltrimethoxysilane, allyltrimethoxysilane or isopropenyltrimethoxysilane;

silanes having a mercapto group, such as mercaptopropyltrimethoxysilane or mercaptoethyltrimethoxysilane;

silanes having an aminoalkyl group, such as (3-aminopropyl)trimethoxysilane, (3-[(2-aminoethyl)amino]propyl)trimethoxysilane, (N, N-dimethylaminopropyl)trimethoxysilane or (N,N-dimethylaminoethyl)trimethoxysilane;

silanes having a haloalkyl group, such as (3-chloropropyl)trimethoxysilane or trifluoropropyltrimethoxysilane;

silanes having a glyceroxy group, such as (3-glyceroxypropyl)trimethoxysilane or di(3-glyceroxypropyl)dimethoxysilane;

silanes having a ureido group, such as (3-ureidopropyl)trimethoxysilane, (3-ureidopropyl)methyidimethoxysilane or (3-ureidopropyl)dimethylmethoxysilane; and

silanes having a cyano group, such as cyanopropyltrimethoxysilane, cyanopropylmethyldimethoxysilane or cyanopropyldimethylmethoxysilane.

In one embodiment of the present disclosure, the compound of formula (IV) comprising a reactive organic group R is chosen from silanes having an epoxy group, silanes having a (meth)acryloyloxy group, silanes having an alkenyl group, silanes having a mercapto group and silanes having an aminoalkyl group.

In another embodiment of the present disclosure, compounds (III) and (IV) can be tetraethoxysilane and methyltrimethoxysilane, respectively.

Use may independently be made, as hydrolysis and polymerization catalysts, of basic catalysts, such as sodium hydroxide, potassium hydroxide, sodium carbonate, sodium hydrogencarbonate or amines (such as ammonia, trimethylamine, triethylamine or tetramethylammonium hydroxide), or acidic catalysts chosen from organic acids, such as citric acid, acetic acid, methanesulphonic acid, p-toluenesulphonic acid, dodecylbenzenesulphonic acid or dodecylsulphonic acid, or inorganic acids, such as hydrochloric acid, sulphuric acid or phosphoric acid. When it is present, the surfactant used can be a nonionic or anionic surfactant or a mixture of the two. Sodium dodecyl-benzenesulphonate can be used as anionic surfactant. The end of the hydrolysis is marked by the disappearance of the products (III) and (IV), which are insoluble in water, and the production of a homogeneous liquid layer.

The condensation stage (b) can use the same catalyst as the hydrolysis stage or another catalyst chosen from those mentioned above.

At the conclusion of this process, a suspension in water of fine organosilicone particles is obtained, wherein the particles can optionally be separated subsequently from the medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

In one embodiment of the present disclosure, the particles obtained (or the spheres) have a mean diameter ranging from 0.05 to 10 μm.

The shape of the portions of hollow spheres obtained according to the above process and their dimensions will depend in particular on the method used to bring the products into contact in stage (b).

A somewhat basic pH and introduction under cold conditions of the polymerization catalyst into the mixture resulting from stage (a) will result in portions of hollow spheres with the shape of round-bottomed “bowls”, whereas a somewhat acidic pH and dropwise introduction of the mixture resulting from stage (a) into the hot polymerization catalyst will result in portions of hollow spheres having a transverse cross section with the shape of a horseshoe.

According to a one embodiment of the present disclosure, portions of hollow spheres with the shape of bowls are used. These can be obtained as disclosed in Japanese Patent Application No. JP-2003-128788.

Portions of hollow spheres with the shape of a horseshoe are also disclosed in Japanese Patent Application No. JP-A-2000-191789.

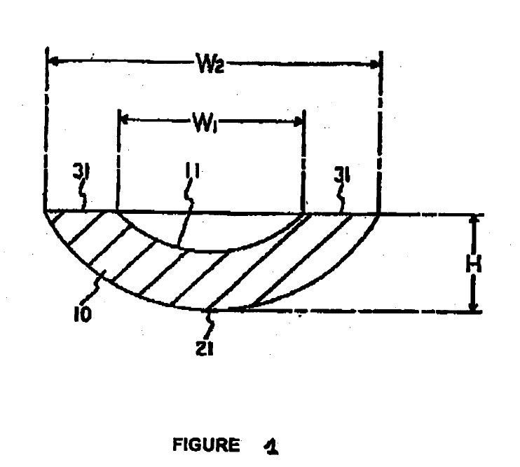

A concave particle formed of portions of spheres with the shape of a bowl is illustrated in transverse cross section in the appended FIG. 1.

As seen in FIG. 1, these concave portions are formed (in longitudinal cross section) of a small internal arc (11), of a large external arc (21) and of segments (31) which connect the ends of the respective arcs. The width (W1) between the two ends of the small internal arc (11) may range from 0.01 to 8 μm, such as from 0.02 to 6 μm, on average. The width (W2) between the two ends of the large external arc (21) may range from 0.05 to 10 μm, such as from 0.06 to 8 μm, on average. The height (H) of the large external arc (21) may range from 0.015 to 8 μm, such as from 0.03 to 6 μm, on average.

The dimensions mentioned above are obtained by calculating the mean of the dimensions of one hundred particles chosen on an image obtained with a scanning electron microscope.

Among the concave particles of portions of spheres which can be used according to the invention, non-limiting mention may be made of:

particles composed of the crosslinked organosilicone TAK-110 (crosslinked methylsilanol/silicate polymer) from Takemoto Oil & Fat, with the shape of a bowl, with a width of 2.5 μm, a height of 1.2 μm and a thickness of 150 nm (particles sold under the name NLK-506 by Takemoto Oil & Fat);

particles composed of the crosslinked organosilicone TAK-110 (crosslinked methylsilanol/silicate polymer) from Takemoto Oil & Fat, with the shape of a bowl, with a width of 2.5 μm, a height of 1.5 μm and a thickness of 350 nm;

particles composed of the crosslinked organosilicone TAK-110 (crosslinked methylsilanovsilicate polymer) from Takemoto Oil & Fat, with the shape of a bowl, with a width of 0.7 μm, a height of 0.35 μm and a thickness of 100 nm; and

particles composed of the crosslinked organosilicone TAK-110 (crosslinked methylsilanol/silicate polymer) from Takemoto Oil & Fat, with the shape of a bowl, with a width of 7.5 μm, a height of 3.5 μm and a thickness of 200 nm.

The concave particles, such as the particles of portions of hollow spheres, can be present in the composition according to the present disclosure, such as in a compact powder, in an amount ranging from 0.01% to 50% by weight relative to the total weight of the composition. In one embodiment of the present disclosure, the concave particles may be present in an amount ranging from 0.1% to 30% by weight relative to the total weight of the composition. In a further embodiment of the present disclosure, the concave particles may be present in an amount ranging from 1% to 15% by weight relative to the total weight of the composition.

The term “pigments” should be understood as meaning white or colored, inorganic or organic particles of any shape which are insoluble in the physiological medium and which are intended to color the composition.

The term “pearlescent agents” should be understood as meaning iridescent particles of any shape, such as particles produced by certain shellfish in their shells or else synthesized.

The pigments can be white or colored, inorganic and/or organic. Among the organic pigments that may be used, non-limiting mention may be made of titanium dioxide, optionally surface treated, zirconium or cerium oxides, as well as zinc, (black, yellow or red) iron or chromium oxides, manganese violet, ultramarine blue, chromium hydrate and ferric blue, or metal powders, such as aluminium powder or copper powder. The pigments can also be chosen from nanopigments formed of metal oxides, such as titanium dioxide, zinc oxide, iron oxide, zirconium oxide, and cerium oxide, and mixtures thereof.

The term “nanopigments” is understood to mean pigments having a mean particle size ranging from 1 nm to 500 nm, such as particle sizes ranging from 10 nm to 100 nm.

Among organic pigments that may be used, non-limiting mention may be made of carbon black, pigments of D & C type and lakes, such as lakes-based on cochineal carmine and on barium, strontium, calcium or aluminium.

The pigments can be present in the composition in an amount ranging from 0.1% to 14.95% by weight relative to the total weight of the composition. In one embodiment of the present disclosure, the pigments can be present in an amount ranging from 0.5% to 12% by weight relative to the total weight of the composition. In another embodiment of the present disclosure, the pigments can be present in an amount ranging from 1% to 10% by weight relative to the total weight of the composition.

The compact powder according to the present disclosure can comprise an additional pulverulent coloring material different from the pigments described above and which can, for example, be chosen from pearlescent agents, glitter and mixtures thereof.

The pearlescent agents can be chosen from white pearlescent agents, such as mica covered with titanium dioxide or with bismuth oxychloride; colored pearlescent agents, such as titanium oxide-coated mica covered with iron oxides, titanium oxide-coated mica covered with ferric blue or chromium oxide, or titanium oxide-coated mica covered with an organic pigment of the abovementioned type; and pearlescent agents based on bismuth oxychloride.

The pearlescent agents can be present in the composition in an amount ranging from 0.1% to 50% by weight relative to the total weight of the composition. In one embodiment of the present disclosure, the pearlescent agents can be present in an amount ranging from 0.1% to 40% by weight relative to the total weight of the composition. In another embodiment, the pearlescent agents can be present in an amount ranging from 0.1% to 30% by weight relative to the total weight of the composition.

The fatty phase of the compact powder, generally referred to as binder, is a fatty phase which is liquid at ambient temperature (25° C.) and can comprise an oil generally used in compact powders.

The oil can be chosen from oils conventionally used as binder in compact powders. Among the additional oils which can be used, non-limiting mention may be made of mink oil, turtle oil, soybean oil, grape seed oil, sesame oil, maize oil, rapeseed oil, sunflower oil, cottonseed oil, avocado oil, olive oil, castor oil, jojoba oil or groundnut oil; hydrocarbon oils, such as liquid paraffins, squalane, liquid petrolatum or polydecene; fatty esters, such as isopropyl myristate, isopropyl palmitate, butyl stearate, isodecyl stearate, hexyl laurate, isononyl isononanoate, 2-ethylhexyl palmitate, 2-hexyldecyl laurate, 2-octyidecyl palmitate, 2-octyldodecyl myristate, 2-octyldodecyl lactate, di(2-ethylhexyl) succinate, diisostearyl malate, glyceryl triisostearate or diglyceryl triisostearate; silicone oils, such as polymethylsiloxanes, polymethylphenylsiloxanes, polysiloxanes modified by fatty acids, fatty alcohols or polyoxyalkylenes, fluorinated silicones or perfluorinated oils; oleic acid, linoleic acid or linolenic acid; or higher fatty alcohols, such as cetanol or oleyl alcohol.

The liquid fatty phase can be present in an amount ranging from 0.1% to 13% by weight relative to the total weight of the composition. In one embodiment of the present disclosure, the liquid fatty phase can be present in an amount ranging from 0.1% to 10% by weight relative to the total weight of the composition. In another embodiment, the liquid fatty phase can be present in an amount ranging from 0.1% to 8% by weight relative to the total weight of the composition.

The composition according to the present disclosure, such as a compact powder, can also comprise fillers.

The term “fillers” should be understood as meaning colorless or white, inorganic or synthetic particles of any shape which are insoluble in the medium of the composition, whatever the temperature at which the composition is manufactured.

The fillers can be inorganic or organic and of any shape, such as platelet, spherical or oblong, whatever the crystallographic form (for example sheet, cubic, hexagonal, orthorhombic, and the like). Non-limiting mention may be made of talc, mica, silica, kaolin, powders formed of polyamide (Nylon®), of poly-β-alanine and of polyethylene, powders formed of tetrafluoroethylene polymers (Teflon®), lauryllysine, starch, boron nitride, polymeric hollow microspheres, such as those of poly(vinylidene chloride)/acrylonitrile, for example Expancel® (Nobel Industrie), or of acrylic acid copolymers, silicone resin microbeads (Tospearls® from Toshiba, for example), particles formed of polyorganosiloxane elastomers, precipitated calcium carbonate, magnesium carbonate, basic magnesium carbonate, hydroxyapatite, barium sulphate, aluminium oxides, polyurethane powders, composite fillers, hollow silica microspheres, glass or ceramic microcapsules, or metal soaps derived from organic carboxylic acids having from 8 to 22 carbon atoms, such as from 12 to 18 carbon atoms, including, for example, zinc stearate, magnesium stearate, lithium stearate, zinc laurate or magnesium myristate.

The fillers can be present in the composition in an amount ranging from 0.1% to 95% by weight relative to the total weight of the composition. In one embodiment of the present disclosure, the fillers can be present in an amount ranging from 1% to 85% by weight relative to the total weight of the composition. In another embodiment of the present disclosure, the fillers can be present in an amount ranging from 1% to 80% by weight relative to the total weight of the composition.

The composition can comprise other ingredients (adjuvants) commonly used in cosmetics, such as waxes, preservatives, cosmetic active principles, moisturizing agents, UV screening agents, thickeners, water, surfactants or fragrances.

Of course, a person skilled in the art will take care to choose the optional adjuvants or adjuvants added to the composition according to the present disclosure such that the beneficial properties intrinsically attached to the composition in accordance with the present disclosure are not, or not substantially, detrimentally affected by the envisaged addition.

The composition according to the present disclosure can be an anhydrous composition, such as a composition comprising less than 2% by weight of water, such as less than 0.5% of water or devoid of water, the water not being added during the preparation of the composition but corresponding to the residual water introduced by the ingredients mixed.

The composition in the form of a compact powder can be prepared by mixing the ingredients of the pulverulent phase (organosilicone particles, fillers and pigments) and by then adding the fatty phase with stirring, the mixture subsequently being milled, sieved, then poured into a dish and compacted.

The milled and sieved mixture of the pulverulent phase and of the fatty phase is compacted using a press, such as by applying a pressure ranging from 0.5 MPa to 10 MPa. In one embodiment, the milled and sieved mixture of the pulverulent phase and of the fatty phase is compacted using a pressure ranging from 1 MPa to 5 MPa.

The composition thus obtained is provided in the form of a compact powder.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The present disclosure is illustrated in more detail by the examples described below.

### COMPARATIVE EXAMPLES 1 to 3

Three compact powders according to the present invention (Example 1) and two compact powders for comparison (Examples 2 and 3) were prepared, these powders having the following compositions:

The contents are expressed as % by weight.

| | Example 1 | Example 2 | Example 3 |
|---|---|---|---|
| | (inventive) | (comparative) | (comparative) |
| Talc | 39.9 | 39.9 | 37.85 |
| Sericite | 33.25 | 33.25 | 31.5 |
| Mica | 4.75 | 4.75 | 4.5 |
| Titanium dioxide | 5.2 | 5.2 | 4.9 |
| Iron oxides | 2.1 | 2.1 | 1.98 |
| Zinc stearate | 0.95 | 0.95 | 0.9 |
| Liquid paraffin | 3.8 | 3.8 | 8.6 |
| Phenyl trimethicone | 4.75 | 4.75 | 4.5 |
| Preservatives | 0.3 | 0.3 | 0.27 |
| Ball-shaped hemispherical | 5 | | 5 |
| particles (1) with a mean | | | |
| width of 2,5 μm, with a | | | |
| thickness of 0.15 μm and | | | |
| with a height of 1.2 μm, | | | |
| composed of the | | | |
| organosilicone* TAK-110 | | | |
| from Takemoto Oil & Fat | | | |
| Spherical polymethyl- | | 5 | |
| silsesquioxane particles | | | |
| (Tospearl 145 B from GE | | | |
| Toshiba Silicone) | | | |

*crosslinked methylsilanol/silicate polymer
(1) particles sold under the name NLK-506 by Takemoto Oil & Fat

The composition of Example 2 (comparative example) comprised polymethylsilsesquioxane particles instead of the crosslinked organopolysiloxane particles present in the composition of inventive Example 1.

The composition of Example 3 (comparative example) comprised a greater amount of oils (liquid binder), i.e. 13.1 g, in comparison with the composition of Example 1, which comprised 8.55 g of oils.

Each composition was prepared by mixing all the powders and by then adding the binder (oils) thereto, this mixture subsequently being milled and sieved until a homogeneous mixture was obtained. 14 g of this mixture were placed in a dish and were then pressed under a pressure of 2 MPa.

Each composition was tested by an expert panel composed of 9 people, the following properties being evaluated when the compact powder was taken with a sponge and when it was applied to the skin:

Amount of product taken with a sponge (disintegration),

Ease of spreading over the skin (slip),

Softness on application, and

Coverage of the product.

The grades assigned for each person range from 0 to 3:

3: excellent,

2: satisfactory,

1: moderate, and

0: poor.

The mean of the grades assigned was calculated.

The mean grades obtained were as follows:

| | Example 1 | Example 2 | Example 3 |
|---|---|---|---|
| Disintegration | 3 | 1 | 0 |
| Slip | 3 | 1 | 0 |
| Softness | 2 | 2 | 1 |
| Coverage | 2 | 1 | 0 |

It was found that only the inventive (Example 1) made it possible to obtain the best properties of disintegration, of ease of application to the skin, of softness and of coverage in comparison with the same properties obtained with the compositions of the prior art (Examples 2 and 3).

1. A cosmetic composition comprising a pulverulent phase and a liquid fatty phase,
wherein the pulverulent phase comprises concave particles and at least one pigment,
wherein the at least one pigment is present in an amount of less than or equal to 15% by weight relative to the total weight of the composition, and
wherein the liquid fatty phase is present in an amount of less than or equal to 13% by weight relative to the total weight of the composition.

2. The composition according to claim 1, wherein the concave particles are in the form of portions of hollow spheres.

3. The composition according to claim 1, wherein the concave particles have a mean diameter ranging from 0.05 μm to 10 μm.

4. The composition according to claim 2, wherein the concave particles in the form of portions of hollow spheres have a transverse cross section with the shape of a horseshoe or arch.

5. The composition according claim 2, wherein the concave particles in the form of portions of hollow spheres are composed of an organosilicone material.

6. The composition according to claim 5, wherein the organosilicone material is a crosslinked polysiloxane with a three-dimensional structure comprising units of formula (I): SiO₂, and of formula (II): R¹SiO_{1.5}, wherein R¹ comprises an organic group having a carbon atom directly connected to the silicon atom.

7. The composition according to claim 6, wherein the organic group is a reactive organic group or an unreactive organic group.

8. The composition according to claim 7, wherein the unreactive organic group is chosen from a C₁-C₄ alkyl group and a phenyl group.

9. The composition according to claim 7, wherein the unreactive organic group is a methyl group.

10. The composition according to claim 7, wherein the reactive organic group is chosen from an epoxy group, a (meth)acryloyloxy group, an alkenyl group, a mercaptoalkyl group, an aminoalkyl group, a haloalkyl group, a glyceroxy group, a ureido group and a cyano group.

11. The composition according to claim 10, wherein the reactive organic group is chosen from an epoxy group, a (meth)acryloyloxy group, an alkenyl group, a mercaptoalkyl and an aminoalkyl group.

12. The composition according to claim 6, wherein R¹ comprises a methyl group.

13. The composition according to claim 6, wherein the organosilicone material comprises the units (I) and (II) according to a unit (I)/unit (II) molar ratio ranging from 30/70 to 50/50.

14. The composition according to claim 13, wherein the organosilicone material comprises the units (I) and (II) according to a unit (I)/unit (II) molar ratio ranging from 35/65 to 45/55.

15. The composition according to claim 5, wherein the organosilicone particles are obtained according to a process comprising:
(a) introducing into an aqueous medium, in the presence of at least one hydrolysis catalyst and optionally of at least one surfactant, a compound (III) of formula SiX₄ and a compound (IV) of formula RSiY₃, wherein
X and Y are chosen from, independently of one another, a C₁-C₄ alkoxy group, an alkoxyethoxy group including a C₁-C₄ alkoxy group, a C₂-C₄ acyloxy group, an N,N-dialkylamino group including a C₁-C₄ alkyl group, a hydroxyl group, a halogen atom and a hydrogen atom, and
R is an organic group comprising a carbon atom connected directly to the silicon atom; and
(b) bringing the mixture resulting from stage (a) into contact with an aqueous solution including at least one polymerization catalyst and optionally at least one surfactant, at a temperature ranging from 30 to 85° C., for at least two hours.

16. The composition according to claim 15, wherein, in stage (a), the molar ratio of the compound (III) to the compound (IV) ranges from 30/70 to 50/50.

17. The composition according to claim 16, wherein, in stage (a), the molar ratio of the compound (III) to the compound (IV) is 40/60.

18. The composition according to claim 15, wherein the ratio by weight of water to the total weight of the compounds (III) and (IV) ranges from 10/90 to 70/30 in stage (a).

19. The composition according to claim 15, wherein the organic group is a reactive organic group or an unreactive organic group.

20. The composition according to claim 19, wherein the unreactive organic group is chosen from a C₁-C₄ alkyl group and a phenyl group.

21. The composition according to claim 19, wherein the unreactive organic group is a methyl group.

22. The composition according to claim 19, wherein the reactive organic group is, chosen from an epoxy group, a (meth)acryloyloxy group, an alkenyl group, a mercaptoalkyl group, an aminoalkyl group, a haloalkyl group, a glyceroxy group, a ureido group and a cyano group.

23. The composition according to claim 22, wherein the reactive organic group is chosen from an epoxy group, a (meth)acryloyloxy group, an alkenyl group, a mercaptoalkyl group and an aminoalkyl group.

24. The composition according to claim 15, wherein R is a methyl group.

25. The composition according to claim 15, wherein the hydrolysis and polymerization catalysts are chosen independently from sodium hydroxide, potassium hydroxide, sodium carbonate, sodium hydrogencarbonate, ammonia, trimethylamine, triethylamine, tetramethylammonium hydroxide, citric acid, acetic acid, methanesulphonic acid, p-toluenesulphonic acid, dodecylbenzenesulphonic acid, dodecylsulphonic acid, hydrochloric acid, sulphuric acid and phosphoric acid.

26. The composition according to claim 1, wherein the concave particles are formed, in longitudinal cross section, of a small internal arc, of a large external arc and of segments which connect the ends of the respective arcs; wherein
the width between the two ends of the small internal arc ranges from 0.01 to 8 μm on average;
the width between the two ends of the large external arc ranges from 0.05 to 10 μm on average; and
the height of the large external arc ranges from 0.015 to 8 μm on average.

27. The composition according to claim 26, wherein the width between the two ends of the small internal arc ranges from 0.02 to 6 μm on average.

28. The composition according to claim 26, wherein the width between the two ends of the large external arc ranges from 0.06 to 8 μm on average.

29. The composition according to claim 26, wherein the height of the large external arc ranges from 0.03 to 6 μm on average

30. The composition according to claim 2, wherein the particles in the form of portions of hollow spheres are present in an amount ranging from 0.01% to 50% by weight relative to the total weight of the composition.

31. The composition according to claim 30, wherein the particles in the form of portions of hollow spheres are present in an amount ranging from 1% to 15% by weight relative to the total weight of the composition.

32. The composition according to claim 1, wherein the at least one pigment is chosen from titanium dioxide, zirconium oxide, cerium oxide, zinc oxides, iron oxides, chromium oxide, manganese violet, ultramarine blue, chromium hydrate, ferric blue, aluminium powder, copper powder, carbon black, pigments of D & C type, and lakes.

33. The composition according to claim 32, wherein the at least one pigment is present in an amount ranging from 0.1% to 14.95% by weight relative to the total weight of the composition.

34. The composition according to claim 33, wherein the at least one pigment is present in an amount ranging from 1% to 10% by weight relative to the total weight of the composition.

35. The composition according to claim 1, further comprising at least one additional coloring material chosen from pearlescent agents, glitter and mixtures thereof.

36. The composition according to claim 35, wherein the pearlescent agents are chosen from mica covered with titanium dioxide or with bismuth oxychloride; titanium oxide-coated mica covered with iron oxides; titanium oxide-coated mica covered with ferric blue or chromium oxide; titanium oxide-coated mica covered with an organic pigment chosen from the said pigments; and pearlescent pigments based on bismuth oxychloride.

37. The composition according to claim 35, wherein the pearlescent agents are present in an amount ranging from 0.1to 50% by weight relative to the total weight of the composition.

38. The composition according to claim 37, wherein the pearlescent agents are present in an amount ranging from 0.1% to 30% by weight relative to the total weight of the composition.

39. The composition according to claim 1, wherein the liquid fatty phase is present in an amount ranging from 0.1% to 13% by weight relative to the total weight of the composition.

40. The composition according to claim 39, wherein the liquid fatty phase is present in an amount ranging from 0.1% to 8% by weight relative to the total weight of the composition.

41. The composition according to claim 1, wherein the liquid fatty phase comprises at least one oil chosen from mink oil, turtle oil, soybean oil, grape seed oil, sesame oil, maize oil, rapeseed oil, sunflower oil, cottonseed oil, avocado oil, olive oil, castor oil, jojoba oil, groundnut oil, liquid paraffins, squalane, liquid petrolatum, polydecene, isopropyl myristate, isopropyl palmitate, butyl stearate, isodecyl stearate, hexyl laurate, isononyl isononanoate, 2-ethylhexyl palmitate, 2-hexyldecyl laurate, 2-octyidecyl palmitate, 2-octyidodecyl myristate, 2-octyldodecyl lactate, di(2-ethylhexyl) succinate, diisostearyl malate, glyceryl triisostearate, diglyceryl triisostearate, silicone oils, fluorinated silicones, perfluorinated oils, oleic acid, linoleic acid, linolenic acid, isostearic acid, cetanol and oleyl alcohol.

42. The composition according to claim 1, further comprising at least one filler.

43. The composition according to claim 42, wherein the at least one filler is chosen from talc, mica, silica, kaolin, powders formed of polyamide, powders formed of poly-β-alanine, powders formed of polyethylene, powders formed of tetrafluoroethylene polymers, lauryllysine, starch, boron nitride, polymeric hollow microspheres, silicone resin microbeads, particles formed of polyorganosiloxane elastomers, precipitated calcium carbonate, magnesium carbonate, basic magnesium carbonate, hydroxyapatite, barium sulphate, aluminium oxides, polyurethane powders, composite fillers, hollow silica microspheres, glass or ceramic microcapsules, and metal soaps derived from organic carboxylic acids having from 8 to 22 carbon atoms.

44. The composition according to claim 42, wherein the fillers are present in an amount ranging from 0.1% to 95% by weight relative to the total weight of the composition.

45. The composition according to claim 44, wherein the fillers are present in an amount ranging from 1% to 80% by weight relative to the total weight of the composition.

46. The composition according to claim 1, further comprising a cosmetic ingredient chosen from waxes, preservatives, cosmetic active principles, moisturizing agents, UV screening agents, thickeners, water, surfactants and fragrances.

47. The composition according to claim 1, wherein the composition is provided in the form of a compact powder.

48. The composition according to claim 1, wherein the composition is in the form of a blusher, an eyeshadow, a face powder, a foundation, a concealer, a product for making up the body, a product for caring for the face, a product for caring for the body or an antisun product.

49. A cosmetic process for making up or for the non-therapeutic care of human keratinous substances comprising
applying to the keratinous substances a composition comprising a pulverulent phase and a liquid fatty phase,
wherein the pulverulent phase comprises concave particles and at least one pigment,
wherein the at least one pigment is present in an amount of less than or equal to 15% by weight relative to the total weight of the composition, and
wherein the liquid fatty phase is present in an amount of less than or equal to 13% by weight relative to the total weight of the composition.

50. The cosmetic process according to claim 49, wherein the keratinous substance is skin.
